(19) United States
(12) Patent Application Publication
(10) Pub. No.: US 2004/0116827 A1
(43) Pub. Date: Jun. 17, 2004

(54) [Cervical Cancer Screening Methods and Apparatus]

(76) Inventor: Osvaldo Antonio Tiberio, Vicente Lopez (AR)

Correspondence Address: OSVALDO ANTONIO TIBERIO, ENTRERIOS 2974, VICENTE LOPEZ, BUENOS AIRES, 1636, AR

(21) Appl. No.: 10/707,135

(22) Filed: Nov. 21, 2003

(30) Foreign Application Priority Data
Nov. 16, 2001 (AR) P010105357

(51) Int. Cl. A61B 10/00

(52) U.S. Cl. 600/569

(57) ABSTRACT

A disposable device for cervical and endocervical cell collection by a subject: female patient, doctor, other health care practitioner (nurse, paramedic, etc.) or any trained person. The object of the present invention is to provide effective methods and apparatus for easy cervical cancer screening which can be administered without the need for direct cervical observation, for health care facilities or without the assistance of health professionals. The present device is easy and inexpensive to manufacture, as well as easy to use and can therefore be made widely available to the consuming public. It may be designed for retail sale as well as mass distribution for mass prevention campaigns (in hospitals, developing countries) This device includes a plastic tube. The distal end of the tube is closed by a very thin membrane (plastic or other similar material). This tube contains a plastic stick with a proximal enlargement for manipulation by the subject. On the distal end of the stick is inserted a bulb-shaped nylon brush. The female patient on her own, the doctor or paramedic personnel, or any trained person perform the cell sample collection according to the instructions detailed above (see claim 11 to 20 and detailed description of the invention)

### BACKGROUND OF INVENTION

Cervical cancer is the second most common cancer among women worldwide, and the leading cause of death from cancer in developing countries.

Annually, about 500.000 new cases of cervical cancer occur worldwide, and 80% of the cases do so in developing countries. These deaths are nearly 100% preventable, and early prevention is the key for the treatment of this disease.

Nowadays, prevention is reached through Papanicolaou (pap) smear. Pap screening is very easy, and only few minutes are necessary to perform it. The health care provider inserts a speculum into the vagina, holding the vaginal walls apart, and takes a sample of endocervical and cervical cells from the cervix and around cervical formix with an instrument (spatula, cotton swab, brush, etc.). This sample is placed on a glass slide, fixed with alcohol or cytological fixative, and sent to the pathologist for conventional analysis.

Nowadays, many types of brushes, spatulas, and cotton swabs are available to perform pap smears. However, these tests must be performed by health care providers (doctors, nurses, paramedics, . . . ), which implies the obligation for female patients to attend medical offices or health care centers.

Unfortunately, all these means of collecting cell samples are insufficient when women fail to comply in scheduling regular visits to health care centers.

Indeed, the major issue remains patient's compliance with the screening. On the one hand, in developed countries, many women do not have regular screening tests for various reasons: lack of time, ignorance, fear, shame, difficult mobility, lack of medical insurance. On the other hand, in developing countries, most women do not have access to health centers.

Consequently, many women are not diagnosed and get to health centers at an advanced stage of the illness. Therefore, their state requires aggressive and expensive treatments, such as invasive surgeries, radiation therapy, chemotherapy, long-term hospitalization, and numerous medical consultations. Furthermore, in most cases, the patient's life expectancy and quality is likely to decrease whereas the cost for public health is increased.

### SUMMARY OF INVENTION

The present invention relates to cervical cancer screening methods and apparatus.

The purpose of this new device is to allow women a better compliance with regular screenings: its use simplifies the pap test procedure and greatly reduces its cost, since medical offices or hospital facilities as well as qualified health personnel are no longer required to perform the endocervical and cervical cell collection. Indeed, in accordance with the present invention, the cell sample collection may be performed by women on their own, health care personnel (ex: nurses), technical health care providers (ex: lab technicians), doctors or any other trained persons. Moreover, the cell sample collection may be performed in the privacy of a home or in regions far off from health care centers.

Thus the use of this device allows to enhance women's compliance with recommended cervical cancer-screening protocols. Indeed, many women who do not comply in scheduling regular visits to health care centers because of their busy way of life may easily perform the test on their own. Furthermore, apart from women who have access to routine tests but do not comply with them, this device can be of use for all women who, for various reasons (fear, shame, ignorance, lack of health insurance or of economic means), do not usually attend health care centers, as well as for elderly female patients with decreased mobility.

Women are thus more likely to perform regular screenings on themselves thanks to the noninvasive, simple, private and economical procedure of the invention.

Furthermore, as the sample is collected without the need for direct cervical observation or access and without the assistance of a doctor or any health care provider, this system is useful for communities whose religious or other believes prevent women from attending health care centers, since one member of these communities may be instructed to perform the sample collection and collect a great amount of samples, later on analyzed in a lab.

The present invention may also be used in mass cervical cancer prevention campaigns, since health care personnel do not need highly complex health facilities to perform the endocervical and cervical cell collection.

In addition, self-screening kits produced in accordance with the present invention may be packaged for retail sale, thus containing the device, alcohol, spray or Thin-prep® (according to every country's specificity), index card for patient's identification and directions for use. Likewise, said invention may be packaged for mass distribution (10, 50, 100, 500 units” boxes), and contain cytological fixative (spray or Thin-prep®, according to every country's specificity), index card for patient's identification and directions for use. These kits would be of great help in developing countries where shortages of doctors prevent most women from receiving screening tests.

It stands to reason that, should the test results be positive or dubious, patients will have to attend health care centers to receive a final diagnosis, according to cervix cancer-detection protocols as established in health care centers.

In developing countries, or regions far off from health care centers, in case a positive result may occur, local health organizations will be responsible for transporting patients to health care centers in order to complete the tests and achieve a full diagnosis.

The present device is easy and inexpensive to manufacture, as well as easy to use and can therefore be made widely available to the consuming public.

Thus, millions of women who do not receive pap tests will have the chance to save their lives, for the benefit of the whole society.

Moreover, early detection, by allowing significant savings in the public health costs, is particularly important in developing countries where health budget is scarce. These savings may then be used on cancer or any other prevention campaign.

### BRIEF DESCRIPTION OF DRAWINGS

Drawing scale: 1=1 cm.

FIGS. 1 & 2 are a series of cross-sectional views of the cell-collection device in accordance with an exemplary embodiment of the present invention.

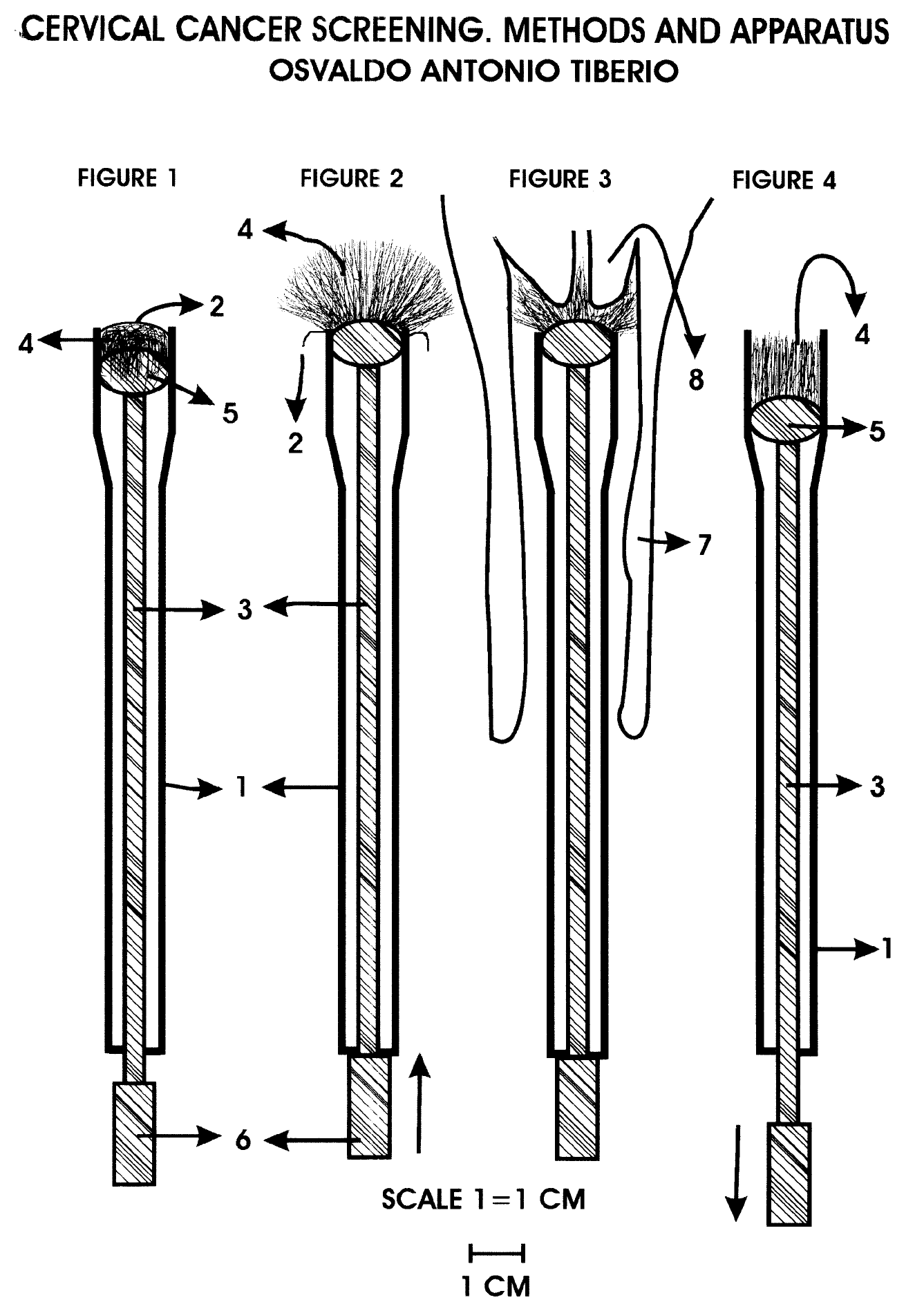

Closed position (FIG. 1): Within the plastic tube (1), the stick (3) is held underneath the nylon membrane (2), which is intact. The tip of the stick (5) is widened so as to receive the brush bristles (4). The proximal end of the stick comprises an enlargement functioning as a stop in opened position (6).

Opened position (FIG. 2): The stick (3) is pushed inside the plastic tube (1). The enlargement of the proximal end of the stick (6) prevents an excessive forward movement that might hurt the cervix. The tip of the stick (5) reaches the top of the distal end of the tube (1). The nylon membrane is broken (2). Brush bristles expand (4).

FIG. 3 is a cross-sectional view of the cell-collection device in accordance with an exemplary embodiment of the present invention, particularly illustrating the device positioned within the vagina (7) and in contact with the cervix (8).

Extraction position (FIG. 4): the stick (3) is pulled back into the plastic tube (1). The brush (4) is totally inserted back in the plastic tube (1) so as to prevent vaginal contamination. The enlarged part of the stick (5) enters in contact with the narrow part of the tube in order to prevent an excessive backward movement. In order to transfer the sample onto the glass slide, the stick is pushed again as in FIG. 2 and the brush again exposed.

### DETAILED DESCRIPTION

The present device basically comprises two parts: A. An external part This part is constituted by a 14 cm long per 1 cm wide plastic tube, the last 3 cm of the distal end being widened 1, 5 cm (this end being introduced into the vagina).

The distal end of this tube is closed by an extra-thin plastic membrane.

This membrane's function is twofold: it avoids the contamination of the brush placed inside the tube while this tube is inserted within the vagina.

it insures the device's dispensability: the membrane being broken, it is impossible to use the device a second time.

The proximal end of the tube has an orifice in which is inserted a stick. This orifice enables the stick's movement to be stable and serves as a stop for the stick so as to prevent its exceeding the edge of the tube and hurting the patient.

B. An internal part: The internal part has a 16, 5 cm long per 4 mm thick internal stick, the last 1 cm of the distal end being widened 1, 3 cm. This end is bulb-shaped and comprises 1 cm long nylon bristles fixed to it. These bristles form a 3, 5 cm diameter bulb-shaped brush which will expand once breaking the membrane and collect the cell sample.

The proximal end is widened to insure easier manipulation and serve as a stop when entering in contact with to the tube to prevent stick's excessive forward movement.

In order to use correctly this device, the health care provider or the female patient herself must follow the following steps: The subject holds the tube and inserts its widest part deeply into the vagina.

Afterwards, the subject pushes the stick in order to break the tube's membrane. The brush will then enter in contact with the cervix.

The subject rotates the stick and consequently the brush. The brush bristles will rub the exocervix and part of the endocervix, thus collecting the cells.

Then the subject gently pulls the stick to insert the brush back into the tube in order to prevent vaginal contamination during the extraction of the tube.

Afterwards, the subject gently pulls the whole device out of the vagina.

Then the subject pushes the stick again in order to expose the brush and transfers the cells sample from the bristles directly into Thin-prep® or onto a glass slide where it will be fixed thanks to a cytological fixative.

Other sample processing and transportation methods can be designed. For instance, the device may be packaged for mass prevention campaigns or hospitals (10, 50, 100, 500 units” boxes), and contain cytological fixative (spray or Thin-prep®, according to every country's specificity), index card for patient's identification and directions for use; likewise the device may be packaged for retail sale, containing alcohol, spray or Thin-prep® (according to every country's specificity), index card for patient's identification and directions for use Thinner apparatus, destined to elderly patients” comfort, can also be designed.

The whole device will be manufactured in a biocompatible material and atraumatically designed to minimize trauma to the vagina and the cervix.

What is claimed is:

1. An Apparatus for the collection of free endocervical and cervical cells by the subject, a doctor or paramedic personnel, said apparatus comprising:
A cylindrical plastic tube, positionable within the vagina;
A cylindrical plastic stick, positioned within the plastic tube.

2. Apparatus as claimed in claim 1 wherein said plastic tube is 14 cm long per 1 cm wide, the last 3 cm of the distal end being widened 1, 5 cm and has a proximal end being ridged so as to be easily handled during use.

3. Apparatus as claimed in claim 1 wherein said plastic tube is closed by an extra-thin plastic membrane having the following functions:
to avoid contamination of the brush (as claimed in claim 6) placed inside said tube while said tube is inserted within the vagina.
to insure the dispensability of said apparatus.

4. Apparatus as claimed in claim 1 wherein said extra-thin plastic membrane is inserted 1 centimeter from the inner end of the tube and is thinner in its central part so as to be easily broken when required, its parts remaining however fixed to the tube's edges (as claimed in claim 1) even when broken.

5. Apparatus as claimed in claim 1 wherein said plastic stick is 16, 5 cm long per 4 mm thick and comprises:
an internal portion;
an external portion.

6. Apparatus as claimed in claim 5 wherein said internal portion has a distal enlargement close to the internal side of the tube (as claimed in claim/1), and comprises a 1,5 cm bulb-shaped nylon brush at its end.

7. Apparatus as claimed in claim 6 wherein said bulb-shaped brush comprises 7 mm long nylon bristles fixed to it, which, before use, are held within the tube (as claimed in claim 1) underneath the plastic membrane (as claimed in claim 3).

8. Apparatus as claimed in claim 6 wherein said bulb-shaped brush has two functions:
to break the plastic membrane (as claimed in claim 3).
to expand once outside the tube (as claimed in claim 1), and enter in contact with the cervix, thus collecting endocervical and cervical cells.

9. Apparatus as claimed in claim 5 wherein said external portion has a proximal end designed to be handled with fingers and pushed inside the plastic tube (as claimed in claim 1) so as to break the membrane (as claimed in claim 3) in order to release the brush (as claimed in claim 6) which expands in a semicircle and, entering in contact with the cervix, will collect the sample.

10. Apparatus as claimed in claim 5 wherein said external portion comprises an enlarged part at its proximal end that, entering in contact with the tube, functions as a stop and prevents excessive forward movement of the stick that might hurt the cervix.

11. A method for self-collecting free endocervical and cervical cells by a subject, medical or paramedical personnel, said method comprising the steps of:
Positioning the cell-collection apparatus (as claimed in claim 1) into the vagina to collect endocervical and cervical cells;
removing said apparatus from the vagina;
transferring the endocervical and cervical cell sample from the brush (as claimed in claim 6) onto a glass slide or into Thin-prep fixing said endocervical and cervical sample on said glass slide with alcohol or cytological fixative;
and sending said glass slide to the pathologist or cytologist for conventional analysis.

12. A method as claimed in claim 11 wherein said positioning step comprises the step of:
inserting the distal end of said apparatus (as claimed in claim 2) deeply within the vagina for a pre-determined cell-collection interval.

13. A method as claimed in claim 11 wherein said positioning step comprises the step of:
pushing the proximal end of the stick (as claimed in claim 9) so that its distal end (as claimed in claim 6) breaks the membrane (as claimed in claim 3) and exposes the brush (as claimed in claim 6) to the cervix uteri.

14. A method as claimed in claim 11 wherein said positioning step comprises the step of:
rotating the proximal end of the stick to collect endocervical and cervical free cells by gently rubbing the cervix and the endocervix.

15. A method as claimed in claim 11 wherein said removing step comprises the step of:
pulling the proximal end of the stick to introduce the brush back into the tube.

16. A method as claimed in claim 11 wherein said removing step comprises the step of:
pulling gently the proximal end of the stick to remove the whole apparatus from the vagina.

17. A method as claimed in claim 11 wherein said transferring step comprises the step of:
pushing again the proximal end of the stick in order to expose the brush again.

18. A method as claimed in claim 11 wherein said transferring step comprises the step of:
transferring the endocervical and cervical cells from the brush onto a glass slide or into Thin-prep®.

19. A method as claimed in claim 11 wherein said fixing step comprises the step of:
fixing the sample on the glass slide with alcohol or cytological fixative or into Thin-prep®.

20. A method as claimed in claim 18 wherein said transferring step comprises the additional step of:
sending the glass slide containing the cell sample or the Thin-prep® receptacle to the pathologist or cytologist for conventional analysis.
